(19) United States
(12) Patent Application Publication
(10) Pub. No.: US 2008/0152724 A1
(43) Pub. Date: Jun. 26, 2008

(54) TREATMENT OF PERIODONTITIS WITH AN INJECTABLE SLOW RELEASE IODINE

(76) Inventor: Mark Hirsh, Wellesley, MA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(21) Appl. No.: 11/963,227

(22) Filed: Dec. 21, 2007

Related U.S. Application Data
(60) Provisional application No. 60/876,414, filed on Dec. 21, 2006.

(51) Int. Cl. A61K 33/18; A61K 33/38; A61P 31/00

(52) U.S. Cl. 424/618; 424/667

(57) ABSTRACT

Compositions for the treatment of periodontitis and methods of use thereof are described. The compositions contain a polymer-iodine complex. The polymer-iodine complex can be suspended in a pharmaceutically acceptable carrier in which the complex is stable and the iodine is stable in its elemental form and/or a combined form (e.g., iodine-iodide complex). In one embodiment, the complex is prepared by the reacting a synthetic or semi-synthetic ionic polymer, with iodine and/or an iodide. The complex will preferably deliver iodine over an extended period of time for example over several days (e.g., at least 3 days, preferably at least 4 days, more preferably at least 5 days, most preferably at least 6 days), preferably at least a week, more preferably at least two weeks. In one embodiment, the polymer-iodine complex forms a gel, alone or by the addition of one or more gelling agents, upon administration into a periodontal pocket. The polymer-iodine complex, or a gel containing the complex can also contain one or more additional active agents and/or pharmaceutically acceptable excipients. The composition is preferably formulated so that it can be administered by injection.

### CROSS-REFERENCE TO RELATED APPLICATIONS

This application claims priority to U.S. Ser. No. 60/876,414 entitled “Treatment of periodontitis With Slow Release Iodine” by Mark Hirsh, which was filed on Dec. 21, 2006. The disclosures in the applications listed above are herein incorporated by reference.

### FIELD OF THE INVENTION

This invention is in the field of compositions for the treatment of periodontal disease and methods of use thereof, particularly compositions containing slow release iodine.

### BACKGROUND OF THE INVENTION

Over 30 million adults in the United States suffer from gingivitis and/or periodontitis, which are major causes of tooth loss. Periodontitis involves multiple infectious agents. Periodontal disease is caused mainly by the accumulation of bacteria (plaque). The destructive toxins and enzymes produced by these bacteria cause the gum tissues to detach and separate from the tooth. The ligaments holding the tooth in its socket break down and the gums pull away from the tooth, resulting in a space between the tooth and gum called the periodontal pocket. Bacterial plaque continues to collect in these pockets, causing them to deepen and also destroying the underlying supporting tissue.

Uncertainty exists as to the exact mechanisms by which the periodontal tissues are destroyed. Many different bacterial taxa are believed to be able to inhabit the periodontal pocket. The abundance and diversity of periodontal pocket microorganisms depend upon several factors, including effectiveness of oral hygiene procedures, pocket depth, degree of gingivitis, flow of crevicular fluid, type of bacteria, and virulence of bacteria. Gram-negative bacteria, gram-positive bacteria and even pseudomonads may also play roles in the pathogenesis of periodontitis. In addition to bacteria, other microorganisms including yeast and other fungi can be involved in periodontitis.

Some periodontal patients lose teeth from periodontal disease despite a regular maintenance schedule which includes plaque control, scaling, and root planing. This is disappointing given the generally low incidence of periodontal breakdown in untreated patients. The data are abundantly clear that the presence of residual periodontopathic bacteria after completion of therapy poses a risk of recurrence of periodontal deterioration. Hence, mechanical removal of calculus and surgical removal of periodontal pockets has been augmented by the use of systemic antibiotics. Systemic antibiotics enter the periodontal tissues and the periodontal pocket via serum (e.g. crevicular fluid) and can affect organisms beyond the reach of cleaning instruments. Early approaches in the use of systemic antibiotics included mainly single drug therapies such as penicillins, tetracycline, metronidazole, and clindamycin. However, use of systemic antibiotics can give rise to a number of adverse reactions and must be administered with care. Besides unwanted side effects and creation of resistant strains, the cost of the antimicrobial drugs must be considered.

Topical antibiotic therapies have also been investigated. However, the effectiveness of such therapies in periodontics is in dispute. Controlled release devices for direct pocket placement containing antibiotics such as tetracycline-HCl, doxycyleine, minocycline, metronidazole, or ofloxacin are commercially available. Most clinical studies have monitored the effect of controlled drug variables characteristic of gingivitis and not necessarily of periodontitis. Generally, the adjunctive or alternative role of topical antibiotic therapies in short- and long-term management of periodontal disease has not been defined. (See, for instance, J. Slots, Selection of Antimicrobial Agents in Periodontal Therapy. J. Periodontal Res., 2002; 37; 389-398.) Hence, while aggressive treatment of periodontitis can give rise to striking clinical outcomes, treatment for chronic periodontitis does not generally provide clear outcomes of disease remission.

Antiseptics have also been explored as possible treatments for periodontitis. An antiseptic is an agent that, applied to living tissues, is able to prevent or arrest the growth or action of microorganisms. Antiseptics have a considerably broader spectrum of activity than antibiotics and, in contrast to antibiotics, often have multiple intracellular targets which reduce the likelihood of resistance development. Antiseptics are limited to use in infected wounds, skin, and/or mucosae, since antiseptics are potentially toxic to the body's cells as well as to infectious agents.

Oral antiseptics should have the following characteristics. They must be active against bacteria, viruses and fungi, and have a reliable effect at the applicable concentrations, typically by absorption onto the oral mucosae. The risk of sensitization must be low. Local irritation and systemic toxic effects should not be present at the application concentration. Finally, the substance preferably should have a neutral taste.

Only a few substances are known to be useful as oral antiseptics. These include povidone-iodine (PVP-I), chlorhexidine, hexetine and hydrogen peroxide (c.f. Rahn, K; Review presentation on povidone-iodine antisepsis in the oral cavity; Postgrad Med J. (1993) 69 (suppl. 3), S4-S9). Rahn has shown that hexetidine was less effective than PVP-I and that chlorhexidine had a greater number of side effects in comparison to PVP-I. Chlorhexidine has an activity that is greater at alkaline pH than at acidic pH. Even low concentrations of chlorhexidine are thought to be toxic to gingival fibroblasts and to reduce the production of collagen, potentially impeding periodontal healing.

Iodine has been used for more than 150 years as a mucosal antiseptic, in the treatment of skin infections and burns, and in wound management. In the 1960s, the introduction of the povidone-iodine complex allowed iodine to be utilized in the eradication of bacterial, fungal, and viral infections. The oxidative potency enables the released iodine to react rapidly with amino acids and unsaturated fatty acids resulting in the destruction of cell structures and enzymes. The antimicrobial spectrum of iodine is effectively universal, including gram-negative and gram-positive bacteria, spores, and mycobacteria, as well as fungi, viruses and protozoa. No development of resistance was determined for PVP-I, in contrast to other antiseptic agents such as chlorhexidine, formaldehyde and benzalkonium chloride. (Lacey R W, Catto A: “Action of povidone-iodine against methicillin-sensitive and resistant cultures of staphylococcus aureus.” Postgraduate Med J., 1993; 69 (suppl 3):78-83). Sodium hypochlorite has many of the properties of an ideal antimicrobial agent, including broad antimicrobial activity, rapid bactericidal action, and relative non-toxicity at the appropriate concentrations. However, it suffers from such disadvantages as loss of activity at low pH and irritation of mucous membranes when used in high concentrations.

There are many studies combining povidone iodine with scaling and root planning which demonstrate that this antiseptic can be statistically effective in reducing total counts of periodontal pathogens. (Hoang T, et al. Povidone-iodine as periodontal pocket disinfection. J. Periodont. Res., 2002; 38; 311-17). In this study, povidone-iodine solution showed high bactericidal activity against all of the test strains after 30 seconds of exposure. In contrast, after the use of chlorhexidine gluconate, residual bacteria were observed in most species. (Kunisada et al, Dermatology, Vol. 195, Suppl. 2 p. 14-18, (1997)). However, it appears that no one has conducted clinical trials of povidone iodine for treatment of periodontitis, perhaps because it is a generic material, and so there is no clinical approval for this use of povidone-iodine.

Iodine has also been encapsulated in polymeric materials, such as chitosan. Chitosan (polyglucosamine; deacetylated chitin) is a natural biocompatible cationic polymer with low toxicity. Chitosan is derived from deacetylation in alkali of chitin, which is the principal component of crustacean shells, such as crab and shrimp. Chitosan is also found in cell walls of bacteria and mushrooms. Chitosan is soluble in dilute acid and water if sufficiently de-acetylated, and can be formed into beads of various sizes. Microspheres of chitosan are known to swell in vivo, and particles of chitosan can be retained on mucosal tissue for significant time periods. Multilayer beads using combinations of chitosan and Ca(2+) as cationic components and alginate and polyphosphates as anions have been tested in many applications for controlled release of drugs. U.S. Pat. No. 5,855,904 reports a biodegradable sustained release preparation for treatment of periodontitis, consisting of antibiotics loaded into calcium alginate microspheres coated by chitosan. However, there does not seem to be clear, independent evidence of efficacy, and the procedure is not used extensively. Kato et al (U.S. Pat. No. 4,275,194) describes a chitosan-iodine amorphous adduct. De Rossa (U.S. Pat. No. 5,538,955) describes a process for preparation of iodine with chitosan in which the complex is made to react in the absence of solvent. Hassan (U.S. Pat. No. 6,521,243) describes a non-staining composition of chitosan and the iodine-iodide complex. Despite the promising properties of chitosan, it is an expensive natural product of variable properties, and may not be the optimal polymer for this application.

Iodine has also been complexed with an ion exchange resin, in a complex sometimes referred to as a cadexomer. It was first reported by Johansson (U.S. Pat. No. 4,010,259), as a complex of iodine with a crosslinked dextran or other gel, and preferably with a charged gel having ion-exchange properties. These materials have been used by Johansson (U.S. Pat. No. 4,783,448) and others to treat skin conditions, including decubitus ulcers.

Despite the fact that iodine products have been known for more than 25 years, the use of an iodine delivery system for treatment of periodontal pockets has not clearly provided a benefit to patients, nor been widely accepted. There exists a need for pharmaceutical compositions which are more effective at treating periodontitis.

Therefore, it is an object of the present invention to provide improved iodine-containing formulations which are more effective for the treatment of periodontitis.

### SUMMARY OF THE INVENTION

Compositions for the treatment of periodontitis and methods of use thereof are described. The compositions contain a polymer-iodine complex.

The polymer-iodine complex can be suspended in a pharmaceutically acceptable carrier in which the complex is stable and the iodine is stable in its elemental form and/or a combined form (e.g., iodine-iodide complex). In one embodiment, the complex is prepared by reacting a synthetic or semi-synthetic ionic polymer, with iodine and/or an iodide.

The complex will preferably deliver iodine over an extended period of time, for example over several days (e.g., at least 3 days, preferably at least 4 days, more preferably at least 5 days, most preferably at least 6 days), preferably at least a week (e.g., at least 7, 8, 9, 10, 11, 12, or 13 days), more preferably at least two weeks. In one embodiment, the polymer-iodine complex forms a gel, alone or by the addition of one or more gelling agents, upon administration into a periodontal pocket. In another embodiment, the complex is biodegradable so that its lifetime in the periodontal pocket is measurable. The polymer-iodine complex, or a gel containing the complex, can further contain one or more additional active agents, such as antimicrobial substances, antiseptic agents, antibiotics and/or one or more pharmaceutically acceptable excipients, such as stabilizers, gel-releasing agents, salts, buffers, and other excipients. The composition is preferably formulated so that it can be administered by injection.

### DETAILED DESCRIPTION OF THE INVENTION

### I. Definitions

“Iodine” or “iodine component”, as used herein, refers to elemental iodine, iodine-iodide complexes, and combinations thereof.

“Complex” or “complexed”, as used herein, describes molecules or ensembles formed by the association of two or more chemical species. The species may be charged, uncharged, or combinations thereof. A complex generally involves a reversible association of molecules, atoms, or ions through weak chemical bonds.

“Biodegradable”, as used herein, means that the polymer can be broken down and absorbed or excreted in a reasonable amount of time under the conditions found at the site of implantation or administration in the body.

### II. Compositions

A. Iodine-Polymer Complexes

1. Iodine and Iodides

The compositions described herein contain iodine and/or iodide complexed to a polymer. Suitable iodides include, but are not limited to, iodine-iodide complexes of the form (cation)⁺(I₃)⁻, where “cation” is a cationic small molecule, such as a metal ion (e.g., potassium or sodium ions), or a cationic group attached to the polymer, and I₃ is the tri-iodide anion. This complex can be formed by mixing an iodide salt, including the ionic iodide complex of a cationic residue, with elemental iodine in solution. The composition is preferably suspended in a pharmaceutically acceptable carrier, such as a non-aqueous solvent.

The concentration of the iodine component ranges from the lower limit of effectiveness to the upper limit of solubility. The concentration of the iodine component depends on the elements of the composition (i.e., the polymer and the pharmaceutically acceptable carrier). A suitable range for most compositions will be in the range of at least 0.05% by weight, preferably at least 0-1%, more preferably at least 0.5%, and ranging upward to 1%, 2%, 3%, 4%, 5% or more. Higher concentrations are preferred when the iodine is stable against aggregation and evaporation during the product's shelf life.

2. Polymers

The compositions contain a polymer which is complexed to the iodine. Suitable materials include, but are not limited to, ion-exchange resins and charged derivatives of synthetic or natural polymers, such as cationic or anionic polymers.

In one embodiment, the polymer may be biodegradable. Biodegradable polymers are well known in the art. In one embodiment, only a portion of a polymer or resin needs to be made of readily degradable bonds to ensure degradation of the entire complex. Suitable biodegradable polymer include, but are not limited to, polymers containing at least a segment selected from polylactides, polyglycolides, polycaprolactones, polyanhydrides, polyamides, polyurethanes, polyesteramides, polyorthoesters, polydioxanones, polyacetals, polycarbonates, polyorthocarbonates, polyphosphazenes, polyhydroxybutyrates, polyhydroxyvalerates, polyalkylene oxalates, polyalkylene succinates, poly(malic acid), poly(amino acids), and copolymers, terpolymers and combinations and mixtures thereof.

In one embodiment, the polymer-iodine complex is not povidone-iodine. The povidone complex of iodine is not substantive to tissue, i.e., it does not at the site of administration when applied topically, and gradually disappears. This is in part due to absorption of the iodine into the tissues, and may be partially due to environment in the body, e.g., physiological pH, rather than the preferred acidic pH for the povidone—iodine complex. Iodine itself is poorly soluble in water, so the solubilizing properties of iodophores are useful even when the complex is not sufficient to prolong the rate of disappearance from a site of application in vivo. However, in treatment of the periodontal pocket, a longer period of delivery is required, because it is not practical to re-administer an iodine complex every day. In addition, the shape of a gingival pocket is often irregular, or even tortuous, making accurate placement of the iodophore difficult unless the complex is reasonably fluid. Thus for treatment of the periodontal pocket with iodine, what is required is a highly stable complex of iodine with a carrier material, which will release therapeutically effective quantities of iodine over an extended period, for example, over a period of several days (e.g., at least 3 days, preferably at least 4 days, more preferably at least 5 days, most preferably at least 6 days), preferably at least a week (e.g., at least 7, 8, 9, 10, 11, 12, or 13 days), more preferably at least two weeks.

i. Ion Exchange Resins

The iodine can be complexed to a water-dispersible, stable ion exchange resin. These complexes are sometimes called “cadexomers”, and have been used in the treatment of decubitus ulcers and the like (e.g., as described in the Merck Index 12th ed., 1999; monograph 1646; or monograph 1610 in the 13^{th} (electronic) edition). Suitable ion exchange resins resins including cationic resins suitable for anion exchange reactions (e.g., DEAE Sephadex) and neutral resins (e.g., Sephadex). The cadexomer-iodine complexes are stable in storage, and release iodine only gradually even after being placed in contact with tissue. Because the resin-iodine complex is much larger than a molecular complex such as povidone-iodine, the rate of washout of cadexomer iodine from a periodontal pocket will be slower than with povidone iodine. Alternatively, the cadexomer iodine can be trapped in the pocket for a prolonged period by use of a gelling agent.

ii. Synthetic or Natural Polymers Containing Cationic Functional Groups

Another class of materials that can bind and stabilize iodine are charged derivatives of synthetic or natural polymers. The polymers contain charged functional groups capable of binding iodine. In one embodiment, the charged group is a cationic group. Exemplary functional groups include, but are not limited to, diethylaminoethyl (“DEAE”) groups and cationic groups containing primary amino groups, secondary amino groups, tertiary amino groups, quaternary amino groups, piperidine groups, pyridine groups, guanidine groups, imidazole groups, and combinations thereof. In one embodiment, the functional group is DEAE. Suitable functionalized polymers include, but are not limited to, DEAE dextran, DEAE cellulose, and DEAE dextrin or DEAE starch.

iii. Cationic Polymers

Other suitable materials that can bind iodine are synthetic cationic polymers. These materials are typically formed by polymerization of cationic monomers or hydrolytically-labile precursors of cationic subunits. Suitable polymers include, but are not limited to, poly(ethyleneimines) and poly(aminoacrylates). The amines in the aminoacrylates are typically tertiary or quaternary. N-acylamino monomers may be polymerized and hydrolyzed to form charged amino groups. Cationic polymers tend to bind negative ligands (including iodine and tri-iodide) more strongly than will neutral (e.g., —OH) groups or anionic groups. Any cationic group is potentially useful, including, but not limited to, cationic groups containing one or more of primary amino, secondary amino, tertiary amino, quaternary amino, piperidine, pyridine, guanidino, imidazole groups, other suitable cationic groups known in the art, and combinations thereof.

B. Gelling and Viscosifying Agents

The polymer-iodine composition may gel alone or in the presence of one or more gelling and/or viscosifying agents. Many of the polymer-iodine complexes are in the form of a dry powder. However, administration of a dry powder to periodontal pockets can be difficult. One alternative is to manipulate the dry powder to form a pre-formed article. The administration of preformed articles can also be difficult, however, as the length, width, and depth of the pocket will differ from patient to patient and may differ among different pockets within the same patient.

One way to overcome these limitations is to administer the compositions as a fluid (e.g., a liquid) or slurry. The volume of flow of gingival crevicular fluid in a periodontal pocket is significant, and turnover of the sulcus (periodontal pocket) contents at least once a day in healthy tissue has been observed (J M Goodwin, Periodontology 2000 Vol. 31, 2003, p 43-54). Therefore, the liquid or slurry should be formulated so that it is retained in the periodontal pocket for an extended period of time after administration.

1. Viscosifying Systems/Agents

One method to increase the retention time of the composition in periodontal pockets is to administer a highly viscous solution. Viscosity of the liquid or slurry can be increased by incorporating a viscosifying agent into the composition and/or minimizing the fluid concentration in the composition. Suitable viscosifying agents include, but are not limited to, sugars, such as sucrose, glucose, maltose dextrose and fructose; hydric alcohols, such as sorbitol, mannitol, xylitol and maltitol; and soluble or partially-soluble polymers such as polydextrose, xanthan gum, guar gum, sodium alginate, carrageenan, hydroxypropyl cellulose (HPC), hydroxypropyl methylcellulose (HPMC), methylcellulose, polyvinylpyrrolidone (PVP), maltodextrin, carbomer, polyvinyl alcohol polyethylene glycol (PEG), polyethylene oxide, carboxymethylcellulose (CMC) and hydroxyethyl cellulose (HEC).

2. Gelling Systems/Agents

The retention time of the fluid or slurry can be increased by causing the composition to form a gel after administration to the periodontal pockets. Gel formation can be induced using a variety of methods. For example, the compositions can be formulated to gel using a mixed solvent system, and optionally a gelling agent. When the more fungible solvent evaporates or otherwise leaves the implant volume, or is replaced by water, the composition itself or the gelling agent gels. Churchill et al U.S. Pat. No. 4,745,160 to Churchill et al. U.S. Pat. No. 5,077,049 to Dunn et al. describe such methods. In the context of the gingival pocket, the dilution of the applied material by secreted fluid can be a method for inducing gelation. A wide variety of biologically-compatible non-aqueous (“organic”) solvents are known, Any material that is liquid at one or more of room temperature and body temperature is suitable, if it is sufficiently low in toxicity and in tissue damage. These include, for example and without limitation, ethanol, propanol, t-butanol, ethylene glycol monomethyl ether, propylene glycol, glycerol, diethylene glycol, dipropylene glycol, liquid polyethylene glycols, liquid polyalkylene glycols comprising propylene glycol monomers, dioxane, dioxanone, ethyl acetate, ethyl lactate, methyl lactate, methyl ethyl ketone (MEK), dimethylformamide, dimethylsulfoxide, tetrahydrofuran, caprolactam, oleic acid, lower alkyl triglycerides, lower alkyl mono- and di-glycerides, silicones, mineral oil, pyrrolidone, N-methylpyrrolidone, and mixtures thereof.

The compositions can also be induced to form a gel by the removal of water. In such a method, water, or a water solution or suspension of a water-soluble or water-swellable polymer, is mixed with the polymer-iodine complex in the form of a dry powder, or a suspension or solution of the powder in a non-aqueous or partially non-aqueous non-solvent for the powder. Redistribution of the water, and/or of the non-solvent, can cause gelation, or at least the formation of a very high viscosity fluid with a long dissipation time. If the two parts are viscous before mixing, and/or a component is slow to swell in water, then there is enough working time to implant the mixture before the gelation is completed.

Finally, a gelling agent can be incorporated into the compositions to induce gel formation. Suitable biocompatible gelling agents are known in the art. The agents may gel upon coming in contact with another material, such as metal ions or an acid or base. Examples of such gelling agents include, but are not limited to, aliginic acid; alginate, such as sodium, potassium, calcium, and ammonium alginates; carrageenan; agar; pectin, locust bean gum, xanthan gum, chitosan; and cellulose derivatives, such as hydroxypropyl methylcellulose and carboxy methylcellulose.

Other suitable gelling agents include those that gel in response to a change in temperature or shearing. Shear-sensitive gels, which can be reversibly made to flow after shearing, and which re-forms a gel on standing are well known in the art. An exemplary system is the interaction of carrageenans with casein. Temperature sensitive gelling agents include, but are not limited to, gelatin and poloxamers.

C. Excipients

Excipients include, but are not limited to, stabilizers, preservatives, coloring agents (e.g. for visualization), taste-masking ingredients, inert salts and/or sugars for tonicity control. When the composition forms a firm gel, the incorporation of pore-forming agents may give better access of the iodine or iodine complex to the tissue from the material.

D. Additional Active Agents

One or more additional active agents can be incorporated into the composition. Suitable active agents include, but are not limited to, antiseptic materials (e.g., silver), antibiotics (local and/or systemic), antimicrobial agents, and anesthetics and analgesics, particularly local anesthetics and analgesics. The one or more additional active agents may be complexed to the polymer or may be dissolved or dispersed in the composition.

### III. Methods of Use

A. Disorders to be Treated

The compositions described herein can be used to treat periodontitis. Periodontitis is the name of a collection of inflammatory diseases affecting the tissues that surround and support the teeth. Periodontitis involves progressive loss of the bone around teeth which may lead to loosening and eventual loss of teeth if untreated. Periodontitis is caused by bacteria that adhere to and grow on tooth surfaces (microbial plaque or biofilms), particularly in areas under the gum line. Periodontitis is very common in most populations but the severe forms of the disease are less common (less than 10% in the U.S.). Dentists diagnose periodontitis by inspecting the tissues around the teeth with a probe and by radiographs to detect bone loss around the teeth. Although the different forms of periodontitis are bacterial diseases, a variety of factors affect the severity of the disease.

Periodontitis is an inflammation of the periodontium—the tissues that support the teeth in the mouth. The periodontium includes the gingiva, or gum tissue, the cementum, or outer layer of the roots of teeth, the alveolar bone, or the bony sockets into which the teeth are anchored, the periodontal ligaments (PDLs), which are the connective tissue fibers that connect the cementum and the gingiva to the alveolar bone.

The primary etiology, or cause, of gingivitis is the accumulation of a bacterial matrix at the gum line, called dental plaque. In some people, gingivitis progresses to periodontitis—the gum tissues separate from the tooth and form a periodontal pocket. Subgingival bacteria (those that exist under the gum line) colonize the periodontal pockets and cause further inflammation in the gum tissues and progressive bone loss. Examples of secondary etiology would be those things that cause plaque accumulation, such as restoration overhangs and root proximity.

The amount of iodine to be administered can be readily determined by one of ordinary skill in the art and is dependent on the age and weight of the patient as well as the disease or disorder to be treated and its severity. The compositions can also contain one or more additional active agents, such as antimicrobial agents, antiseptic agents, and combinations thereof.

### IV. Methods of Administration

The iodine/polymer complex may be supplied with or in a specialized applicator. The device will have an outlet for the solution, an ejector for expelling the solution through the outlet and a hollow tube fitted to the outlet for inserting the solution into a site of the body such that the solution can be applied to the desired site. The applicator will be sterile, and preferably disposable.

In one embodiment the applicator is a conventional syringe, with a length of tubing pre-connected to the outlet of the syringe, prefilled with a fluid component containing the polymer-iodine complexed in a suitable suspending medium.

In another embodiment, the applicator will be a syringe containing a pre-measured amount of iodine complex, which will be reconstituted with a fixed amount of a supplied fluid to make a dispensible suspension or solution.

In still another embodiment, the applicator is formed of a squeezable plastic, and has a long tip, narrow at the exit end, attached to a hollow squeezable form. The applicator is filled by squeezing it to expel air, and allowing it to take up the iodine complex from a vial. The complex, which is preferably suspended in a fluid, the fluid being applied either at the time of dispensation, or from a separate container, is then dispensed into the periodontal pockets to be treated. The applicator may have a small effective internal volume, so that the amount of iodine complex and carrier will somewhat overfill a typical sulcus, for example having an effective volume less than about 1 ml, preferably less than about 0.5 ml. The person applying the complex squeezes the applicator gently to dispense sufficient material to fill the sulcus, after which the applicator can be refilled with fluid and applied again.

### V. Kits

The compositions can be packaged in a kit which contains a means for administering the composition, such as a syringe, and instructions for administering the composition. In one embodiment, the iodine-polymer complex may be formulated in a pharmaceutically acceptable carrier and optionally containing a gelling agent. In another embodiment, the iodine-polymer complex may be packaged as a dry powder which is suspended in a pharmaceutically acceptable carrier and optionally a gelling agent prior to administration.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Zone of Inhibition Test of Cadexomer-Iodine Particles (Powder) and Gels

The efficacy of an iodophor complex against microorganisms typically found in periodontitis was evaluated. Cadexomer-iodine powder and gels were evaluated against Pseudomonas aeruginosa, ATCC #33400, Staphylococcus aureus, ATCC #6538; and Candida albicans, ATCC #10231.

A 18-24 hour culture of each test microorganism was prepared. Bacterial isolates were grown in Trypticase Soy Broth (TSB) at 30-35° C. Candida albicans was incubated at 20-25° C. in Sabauraud Dextrose Broth (SDB) for 48 hours. As a control, for validity, all negative media controls must show no growth, and all test microorganisms must show viable growth.

### Test Method: Zone of Inhibition (Agar Plate Method)

1. Duplicate Trypticase Soy Agar (TSA) plates was inoculated with each bacterial test microorganism. Each plate was inoculated by adding 0.1 ml of test microorganism from an 18-24 hour culture. The microorganisms were spread evenly over the plate using a sterile cotton swab to create an even layer of bacteria.
2. Procedure 1 was repeated using Sabauraud Dextrose Agar (SAB) plates for Candida albicans.
3. Using a sterile core borer, a hole (well) was cut in the center of each agar plate and the agar plug was removed.
4. 100 mg of test sample was added to the well, followed by a wetting using 200 μl of sterile water.
5. The TSA plates were incubated for 48 hours at 30-35° C. The SAB plates were incubated for 5 days at 20-25° C.
6. After incubation, the zone of inhibition around the well of each plate was measured using a caliper.

### Results:

### Diameter of Zone of Inhibition of Microbial Growth by Cadexomer-Iodine

| | | Cadexomer-Iodine |
|---|---|---|
| Microorganism | Cadexomer-Iodine Powder | Ointment |
| P. aeruginosa | 1.375 cm | 0.720 cm |
| S. aureus | 0.955 cm | 0.575 cm |
| C. albicans | 2.065 cm | 1.200 cm |

The results demonstrate that iodine is liberated both from cadexomer-iodine powder and, at a slower rate or effective concentration, from an ointment containing cadexomer-iodine powder in an ointment base. The iodine was effective in inhibiting the growth of different types of organisms, including both bacteria and fungi.

Other materials to be evaluated include iodine complexes with DEAE-functionalized polymers. Methodologies for how these materials can be prepared are provided below.

DEAE Dextran

DEAE Dextran, a water-soluble linear DEAE polymer, will be obtained from a commercial supplier, such as Sigma-Aldrich. The polymer will be mixed with an approximately equal volume of propylene glycol (a non-ionic non-aqueous solvent). After several minutes, the mixture will become clear and highly viscous, with some elastic character and shear-thinning. A sufficient amount of water will be added to dissolve the polymer. The mixture can be placed in a periodontal pocket, and should gel as crevicular fluid is excreted. A commercial product would further contain active iodine (elemental iodine optionally with iodide salts).

DAEA Sephadex

DEAE Sephadex, a crosslinked ion exchange resin available from Amersham, will be dispersed in about an equal volume of glycerol. The resulting slurry will be mixed with an approximately equal volume of water. The amount of water will be substantially less than the equilibrium water absorption of the DE-Sephadex, which typically swells 3-5 times in water. The resin is expected to form a viscous slurry, which tends to self-adhere. The addition of 1 volume of water is not expected to decrease the viscosity, but the addition of 5 volumes of water is likely to decrease the viscosity/gelation. Loading of the resin with active iodine is expected to shift the amounts of solvents required to achieve the effects. Iodine-loaded slurries can be placed in periodontal pockets and liberate iodine to treat periodontitis.

Carrageenan

Powdered kappa-carrageenan is loaded with iodine by suspension of the carrageenan particles in an ethanol solution of iodine for at least four hours. The treated gum is extracted and dried. When taken up in a 1:1 (v/v) ethanol-water mixture and implanted in a periodontal pocket, the complex gels under the influence of potassium and calcium ions in the crevicular fluid, delivering iodine over a prolonged period of time.

I claim:

1. A method for the treatment of an infection in a periodontal pocket, the method comprising:
administering to a patient in need thereof an effective amount of an composition comprising an iodine component complexed to a polymer, and a pharmaceutically acceptable carrier,
wherein the composition provides sustained release of the iodine component.

2. The method of claim 1 wherein the iodine component is selected from the group consisting of iodine (I₂), iodide (I⁻), tri-iodide (iodine/iodide complex; I₃⁻), and combinations thereof.

3. The method of claim 1, wherein the polymer is cationic.

4. The method of claim 1, wherein the polymer is anionic.

5. The method of claim 1 wherein the pharmaceutically acceptable carrier comprises a non-aqueous water-miscible solvent.

6. The method of claim 5, wherein the water-miscible non-aqueous solvent is selected from ethanol, propanol, t-butanol, ethylene glycol monomethyl ether, propylene glycol, glycerol, diethylene glycol, dipropylene glycol, liquid polyethylene glycols, liquid polyalkylene glycols comprising propylene glycol monomers, dioxane, dioxanone, ethyl acetate, ethyl lactate, methyl lactate, methyl ethyl ketone (MEK), dimethylformamide, dimethylsulfoxide, tetrahydrofuran, caprolactam, oleic acid, lower alkyl triglycerides, lower alkyl mono- and di-glycerides, silicones, mineral oil, pyrrolidone, N-methylpyrrolidone, and mixtures thereof.

7. The method of claim 1, wherein the viscosity of the polymer increases or the polymer forms a gel upon contact with bodily fluids.

8. The method of claim 1, wherein the composition further comprises a gelling agent which gels upon administration to the periodontal pocket.

9. The method of claim 8, wherein the gelling agent gels upon partial replacement of a non-aqueous solvent with an aqueous solvent.

10. The method of claim 8, wherein the gelling agent gels upon contact with a bodily fluid.

11. The method of claim 2, wherein the composition further comprises a metal cation.

12. The method of claim 8, wherein the composition further comprises a metal cation.

13. The composition of claim 1 further comprising silver.

14. The composition of claim 1 further comprising silver iodide.

15. The method of claim 3, wherein the cationic group comprises one or more groups selected from the group consisting of primary amino, secondary amino, tertiary amino, quaternary amino, piperidine, pyridine, guanidino, imidazole groups, and combinations thereof.

16. The method of claim 15, wherein the cationic group is diethylaminoethyl (DEAE).

17. The method of claim 1, wherein the polymer is biodegradable.

18. The method of claim 17, wherein the polymer contains one or more segments selected from the group consisting of polylactides, polyglycolides, polycaprolactones, polyanhydrides, polyamides, polyurethanes, polyesteramides, polyorthoesters, polydioxanones, polyacetals, polycarbonates, polyorthocarbonates, polyphosphazenes, polyhydroxybutyrates, polyhydroxyvalerates, polyalkylene oxalates, polyalkylene succinates, poly(malic acid), poly(amino acids) and proteins, polysaccharides, and copolymers, terpolymers and combinations and mixtures thereof.

19. The method of claim 1, wherein the periodontal pocket is root-planed prior to the administration of the composition.

20. The method of claim 1, wherein the composition release iodine for a period of at least one week.

21. The method of claim 20, wherein the composition release iodine for a period of at least two weeks.

22. A pharmaceutical composition for the treatment of an infection a periodontal pocket comprising
an effective amount to treat an infection in the periodontal pocket of an iodine component complexed to a biocompatible polymer,
and a physiologically acceptable carrier,
wherein the composition provides sustained release of the iodine component.

23. The composition of claim 22, wherein the iodine component is selected from the group consisting of iodine (I₂), iodide (I⁻), tri-iodide (iodine/iodide complex, I₃⁻), and combinations thereof.

24. The composition of claim 22, wherein the polymer is cationic.

25. The composition of claim 22, wherein the polymer is anionic.

26. The composition of claim 22, wherein the pharmaceutically acceptable carrier comprises a non-aqueous water-miscible solvent.

27. The composition of claim 26, wherein the water-miscible non-aqueous solvent is selected from ethanol, propanol, t-butanol, ethylene glycol monomethyl ether, propylene glycol, glycerol, diethylene glycol, dipropylene glycol, liquid polyethylene glycols, liquid polyalkylene glycols comprising propylene glycol monomers, dioxane, dioxanone, ethyl acetate, ethyl lactate, methyl lactate, methyl ethyl ketone (MEK), dimethylformamide, dimethylsulfoxide, tetrahydrofuran, caprolactam, oleic acid, lower alkyl triglycerides, lower alkyl mono- and di-glycerides, silicones, mineral oil, pyrrolidone, N-methylpyrrolidone, and mixtures thereof.

28. The composition of claim 22, wherein the viscosity of the polymer increases or the polymer forms a gel upon contact with bodily fluids.

29. The composition of claim 22, wherein the composition further comprises a gelling agent which gels upon administration to the periodontal pocket.

30. The composition of claim 29, wherein the gelling agent gels upon partial replacement of a non-aqueous solvent with an aqueous solvent.

31. The composition of claim 29, wherein the gelling agent gels upon contact with a bodily fluid.

32. The composition of claim 23, wherein the composition further comprises a metal cation.

33. The composition of claim 29, wherein the composition further comprises a metal cation.

34. The composition of claim 22 further comprising silver.

35. The composition of claim 22 further comprising silver iodide.

36. The composition of claim 24, wherein the cationic group comprises one or more groups selected from the group consisting of primary amino, secondary amino, tertiary amino, quaternary amino, piperidine, pyridine, guanidino, imidazole groups, and combinations thereof.

37. The composition of claim 36, wherein the cationic group is diethylaminoethyl (DEAE).

38. The composition of claim 22, wherein the polymer is biodegradable.

39. The composition of claim 38, wherein the polymer contains one or more segments selected from the group consisting of polylactides, polyglycolides, polycaprolactones, polyanhydrides, polyamides, polyurethanes, polyesteramides, polyorthoesters, polydioxanones, polyacetals, polycarbonates, polyorthocarbonates, polyphosphazenes, polyhydroxybutyrates, polyhydroxyvalerates, polyalkylene oxalates, polyalkylene succinates, poly(malic acid), poly(amino acids) and proteins, polysaccharides, and copolymers, terpolymers and combinations and mixtures thereof.

40. The composition of claim 22, wherein the composition releases the iodine component for a period of at least one week.

41. The composition of claim 40, wherein the composition releases iodine for a period of at least two weeks.

42. A kit for the treatment of an infection in the periodontal pocket, the kit comprising a composition comprising an effective amount of an iodine component for treating the infection complexed to a polymer, a pharmaceutically acceptable carrier, and a means for administering the composition into the periodontal pocket.
